(19) United States
(12) Patent Application Publication
(10) Pub. No.: US 2009/0087497 A1
(43) Pub. Date: Apr. 2, 2009

(54) Compositions and methods for treating symptoms of aging

(75) Inventor: Giovanni Paternostro, La Jolla, CA (US)

Correspondence Address: BIO TECHNOLOGY LAW GROUP;C/O PORTFOLIOIP, P.O. BOX 52050, MINNEAPOLIS, MN, 55402, US

(73) Assignee: Burnham Institute for Medical Research, La Jolla, CA (US)

(21) Appl. No.: 12/228,993

(22) Filed: Aug. 18, 2008

Related U.S. Application Data
(60) Provisional application No. 60/965,032, filed on Aug. 16, 2007.

(51) Int. Cl. A61K 33/30; A61P 43/00

(52) U.S. Cl. 424/641

(57) ABSTRACT

Compositions and methods are provided that are based on a discovery that a combination of three compounds, doxycycline, selenium, and zinc, retards physiological age-related changes (for example cardiac aging and the decline in exercise capacity) and can also prolong survival.

### CROSS-REFERENCE TO RELATED CASES

This application claims priority from U.S. provisional patent application Ser. No. 60/965,032, filed Aug. 16, 2007, which is incorporated herein by reference in its entirety.

### STATEMENT OF GOVERNMENT RIGHTS

The invention was supported, at least in part, by a grant from the Government of the United States of America (grant no. R21AG026729 2082902 from the National Institutes of Health. The Government has certain rights to the invention.

### BACKGROUND

The number of genes and interventions affecting aging is large: more than 200 aging genes are listed in the Sage database (Strauss and LaMarco, Exp. Gerontol. 37:1297-1301, 2002). In a systematic study in C. elegans Lee et al (Nat. Genet. 33:40-48, 2003) could show that interventions on 2% of genes in chromosome I caused a reproducible prolongation of lifespan. Furthermore, the effects reported depend on the quantitative expression of the genes (Lin et al., Sience 282:943-946, 1998; Rogina et al., Science 290:2137-2140, 2000) and on the genetic background (Spencer et al., Aging Cell 2:123-130, 2003; Helfand and Rogina, Bioessays 25:134-141, 2003). QTL mapping experiments in Drosophila also support the view of longevity as an extremely complex quantitative trait (Mackay, Mech. Aging Dev. 123:95-104, 2002). Therefore we cannot expect to “cure” aging as we might do with some diseases, but rather to find a progressively larger number of interventions that might delay some of its manifestation, as it has been done in many model organisms. This makes it an ideal subject for the use of combined interventions.

The most marked alterations during aging derive from diminished functional reserve and inadequate response to stress (Taffet, “Physiology of Aging,” In: Geriatric Medicine, ed. Cassel [New York: Springer], 2002, pp. 27-35), both in quantitative and in qualitative terms (the expression “loss of complexity” has been used [Lipsitz, Sci. Aging Knowledge Environ. 2004:pe16, 2004]). This is shown, for example, by the changes with age in maximal heart rate after exercise stress.

Exercise capacity represents a physiological summary of body function. It has a large influence on quality of life in older subjects, and can easily be measured in humans (Guralnik et al., New Engl. J. Med. 332:556-561, 1995). It is also severely compromised in frail individuals, which are considered clinically as aging at a faster rate (Ferrucci et al., J. Endocrinol. Invest. 25(10 Suppl.):10-15, 2002; Fried and Walston, “Frailty and failure to thrive,” In: Principles of Geriatric Medicine and Gerontology, ed. Hazzard, 5^{th} ed. [New York: McGraw-Hill], 2003, pp. 1487-1502).

There is a need for therapies that reduce or retard physiological age-related changes or prolong survival. The present inventions meets this and other needs.

### SUMMARY OF THE INVENTION

We have discovered that a combination of three compounds, doxycycline, selenium, and zinc, retards physiological age-related changes and improves age-related dysfunction (for example cardiac aging and the decline in exercise capacity) and can also prolong survival.

Accordingly, the present invention provides compositions and methods for treating a symptom of aging, improving an age-related dysfunction or prolonging survival in a mammal in need of such treatment.

According to one embodiment of the invention, method are provided for treating a symptom of aging, improving an age-related dysfunction or prolonging survival in a mammal in need of such treatment comprising administrating to the mammal an effective amount of a composition comprising doxycycline, selenium and zinc. According to one embodiment, the symptom of aging is, for example, cardiac aging or a decline in exercise capacity. According to another embodiment, the composition comprises about 125 μg/kg body weight per day to about 5.00 mg/kg body weight per day of doxycycline (or about 10 to 400 mg per day for a human). According to another embodiment, the composition comprises about 0.125 μg/kg body weight per day to about 5.00 μg/kg body weight per day of selenium (or about 10 to 400 μg per day for a human). According to another embodiment, the composition comprises about 12.5 μg/kg body weight per day to about 500 μg/kg body weight per day of zinc (or about 1 to 40 mg per day for a human). According to another embodiment, the composition comprises about 125 μg/kg body weight per day to about 5.00 mg/kg body weight per day of doxycycline, about 0.125 μg/kg body weight per day to about 5.00 μg/kg body weight per day of selenium, and about 12.5 μg/kg body weight per day to about 500 μg/kg body weight per day of zinc. According to another embodiment, the mammal treated is a human.

According to another embodiment of the invention, a method is provided for making a medicament useful in treating a symptom of aging, improving an age-related dysfunction or prolonging survival in a mammal in need of such treatment, the method comprising incorporation of doxycycline, selenium and zinc into a pharmaceutical composition comprising the doxycycline, selenium and zinc and a pharmaceutically acceptable carrier.

According to another embodiment of the invention, compositions are provided for treating a symptom of aging, improving an age-related dysfunction or prolonging survival in a mammal in need of such treatment. Such compositions comprise doxycycline, selenium and zinc. According to another embodiment, the symptom of aging is selected from the group consisting of cardiac aging and a decline in exercise capacity. According to another embodiment, the composition comprises about 10 mg to about 400 mg of doxycycline. According to another embodiment, the composition comprises about 10 μg to about 400 μg of selenium. According to another embodiment, the composition comprises about 1 mg to about 40 mg of zinc. According to another embodiment, the composition comprises about 10 mg to about 400 mg of doxycycline, about 10 μg to about 400 μg of selenium, and about 1 mg to about 40 mg of zinc. According to another embodiment, the composition comprises a pharmaceutically acceptable carrier. The foregoing dosages for the compositions of the invention are daily dosage units for humans. Smaller dosage units may be used for administration more than once per day. Also, for treatment of non-human mammals, the dosage would be adjusted to provide the proper dose per kg body weight.

The foregoing and other aspects of the invention will become more apparent from the following detailed description, accompanying drawings, and the claims.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below.

### BRIEF DESCRIPTION OF THE FIGURES

The patent or application file contains at lease two drawings executed in color. Copies of this patent or patent application publication with color drawings, will be provided by the Office upon request and payment of the necessary fee.

FIG. 1 shows variation with age of heart rate measured in Drosophila before and during the stress of increased temperature and in humans at exhaustion during upright, seated cycle ergometry. From Lakatta (Circulation Res. 88:984-986, 2001), combining data from Patemostro et al. (Circulation Res. 88:1053-1058, 2001) and Fleg et al. (J. Appl. Physiol. 78:890-900, 1995). Heart rate expressed in beats per minute.

FIG. 2 shows a summary of the fully factorial dataset. Doses used for each drug are indicated (see the legend; also see Example 1 below for exact doses, the highest number in the legend indicates the highest dose). The number on the right of each combination is a summary score (z-score) obtained from the three phenotypes we measure: maximal heart rate, exercise capacity and survival, in aged flies. Scores are ordered in descending order, with the best on top. The four columns show, from left to right, the effects of using 1, 2, 3 and 4 drugs in various combinations. The arrow on the right shows the value of the score in control, untreated flies of the same age. The effects do not appear to be additive but rather complex interactions are present.

FIG. 3 shows the maximal heart rate and the maximal climbing velocity at 30 days of age are significantly (using one-way ANOVA and the Bonferroni correction) increased in the same four treatment groups, compared to controls of the same age.

### DETAILED DESCRIPTION OF THE INVENTION

The invention provides compositions and methods for retarding or reducing physiological age-related changes (for example cardiac aging and decline in exercise capacity) and/or prolonging survival.

### DEFINITIONS

As used herein, “agent” refers to any substance that has a desired biological activity. An “anti-aging agent” has detectable biological activity in treating physiological age-related changes (for example cardiac aging and decline in exercise capacity) and/or prolonging survival, in a host.

As used herein, “effective amount” refers to an amount of a composition that causes a detectable difference in an observable biological effect, for example, a statistically significant difference in such an effect. The detectable difference may result from a single substance in the composition, from a combination of substances in the composition, or from the combined effects of administration of more than one composition. For example, an “effective amount” of a composition comprising doxycycline, selenium, and zinc may refer to an amount of the composition that retards or reduces physiological age-related changes or dysfunction (for example cardiac aging and decline in exercise capacity) and/or prolongs survival, or another desired effect in a host. A combination of doxycycline, selenium, and zinc and another substance, e.g., an anti-aging agent, or other active ingredient, in a given composition or treatment may be a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition.

As used herein, the term “patient” refers to organisms to be treated by the compositions and methods of the present invention. Such organisms include, but are not limited to, “mammals,” including, but not limited to, humans, monkeys, dogs, cats, horses, rats, mice, etc. In the context of the invention, the term “subject” generally refers to an individual who will receive or who has received treatment (e.g., administration of a compound of the invention, and optionally one or more anticancer agents) for cancer.

As used herein, “pharmaceutically acceptable salts” refer to derivatives of doxycycline, selenium, and zinc or other disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of doxycycline, selenium, and zinc or other compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., p. 1418 (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

One diastereomer of a compound may display superior activity compared with the other. When required, separation of the racemic material can be achieved by HPLC using a chiral column or by a resolution using a resolving agent such as camphonic chloride as in Thomas J Tucker, et al., J. Med. Chem. 1994 37, 2437-2444. A chiral compound may also be directly synthesized using a chiral catalyst or a chiral ligand, e.g. Mark A. Huffman, et al., J. Org. Chem. 1995, 60, 1590-1594.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

The compounds described herein can be administered as the parent compound, a pro-drug of the parent compound, or an active metabolite of the parent compound.

“Pro-drugs” are intended to include any covalently bonded substances which release the active parent drug or other formulas or compounds of the present invention in vivo when such pro-drug is administered to a mammalian subject. Pro-drugs of a compound of the present invention are prepared by modifying functional groups present in the compound in such a way that the modifications are cleaved, either in routine manipulation in vivo, to the parent compound. Pro-drugs include compounds of the present invention wherein the carbonyl, carboxylic acid, hydroxy or amino group is bonded to any group that, when the pro-drug is administered to a mammalian subject, cleaves to form a free carbonyl, carboxylic acid, hydroxy or amino group. Examples of pro-drugs include, but are not limited to, acetate, formate and benzoate derivatives of alcohol and amine functional groups in the compounds of the present invention, and the like.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic.

Obviously, numerous modifications and variations of the present invention are possible in light of the above teachings. It is therefore to be understood that within the scope of the appended claims, the invention may be practiced otherwise than as specifically described herein.

The compositions of the invention can be formulated as pharmaceutical compositions and administered to a mammalian host, such as a human patient, in a variety of forms adapted to the chosen route of administration, i.e., orally or parenterally, by intravenous, intramuscular, topical or subcutaneous routes.

Such compositions may be systemically administered in vivo by a variety of routes. For example, they may be administered orally, in combination with a pharmaceutically acceptable excipients such as an inert diluent or an assimilable edible carrier. They may be enclosed in hard or soft shell gelatin capsules, may be compressed into tablets, or may be incorporated directly with the food of the patient's diet. For oral administration, the active ingredient or ingredients may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active ingredient in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compositions may also be administered intravenously or intraperitoneally by infusion or injection. Solutions of the cyclosporin, its salts and other active ingredients can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating cyclosporin A or other active ingredients in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, cyclosporin A and other active ingredients may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of doxycycline, selenium, zinc and other active ingredients can be determined by comparing their in vitro activity and in vivo activity in animal models. Methods for the extrapolation of effective dosages in mice, and other animals, to humans are known to the art; for example, see U.S. Pat. No. 4,938,949.

Generally, the concentration of cyclosporin or other active ingredients of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use alone or with other anticancer compounds will vary not only with the particular salt selected but also with the route of administration, the nature of the condition being treated and the age and condition of the patient and will be ultimately at the discretion of the attendant physician or clinician.

In general, however, a suitable dose may be in the range of from about 0.5 to about 100 mg/kg, e.g., from about 1 to about 75 mg/kg of body weight per day, or 1.5 to about 50 mg per kilogram body weight of the recipient per day, or about 2 to about 30 mg/kg/day, or about 2.5 to about 15 mg/kg/day.

The compound may be conveniently administered in unit dosage form; for example, containing 5 to 1000 mg, conveniently 10 to 750 mg, most conveniently, 50 to 500 mg of active ingredient per unit dosage form.

The active ingredient may be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The following invention will be further described by the following nonlimiting example.

### EXAMPLE 1

We have discovered that a combination of three compounds, doxycycline, selenium, and zinc, retards physiological age-related changes (for example cardiac aging and the decline in exercise capacity). It can also prolong survival. This discovery derives from a large screen of individual and combined compounds performed in Drosophila melanogaster. The end points measured were: decline in maximal heart rate with age, decline in maximal exercise capacity and survival.

The Drosophila heart model. The cardiac functional measurement obtained from the Drosophila model that is better suited for future studies in mice and humans is the decline of maximum heart rate with age. This is a very important index of cardiac function. It is one of the main causes of the decrease in the capacity for physical work in older people (Lakatta, Heart Fail. Rev. 7:5-8, 2002). The other determinant of cardiac output, the stroke volume (the amount of blood pumped with each heart beat), does not change with age (Lakatta, Heart Fail. Rev. 7:5-8, 2002). This age-related change is also present in rodents (Corre et al., J. Appl. Physiol. 40:741-744, 1976; Bernard et al., J. Appl. Physiol. 36:472-474, 1974) and it is easily measured in rodents and humans using non-invasive methods (Corre et al., J. Appl. Physiol. 40:741-744, 1976; Bernard et al., J. Appl. Physiol. 36:472-474, 1974; Fleg et al., J. Appl. Physiol. 78:890-900, 1995).

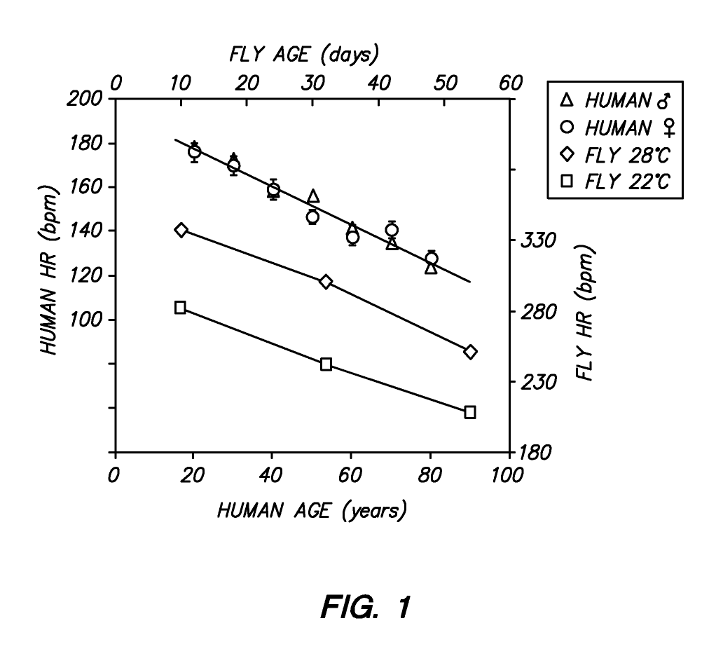

A detailed account of the Drosophila cardiac aging model has been presented in our previous publication (Paternostro et al., Circulation Res. 88:1053-1058, 2001). We developed new methods for imaging rapidly and non-invasively the adult Drosophila heart and for automated processing of the images obtained. We can measure heart rate and its variability. We can also obtain measurements of end-systolic and end-diastolic dimensions. We have previously shown differences in cardiac function between young and aged fly hearts that mimic those observed in humans. FIG. 1 shows variation with age of heart rate measured in Drosophila before and during the stress of increased temperature and in humans at exhaustion during upright, seated cycle ergometry. From Lakatta (Circulation Res. 88:984-986, 2001), combining data from Paternostro et al. (Circulation Res. 88:1053-1058, 2001) and Fleg et al. (J. Appl. Physiol. 78:890-900, 1995).

Exercise capacity measurements. We have recently developed a method to measure exercise capacity in Drosophila, which we have used in all the measurements described in the following section. We designed an apparatus in which to measure climbing velocity to assess overall fly fitness and exercise capacity and its changes with age. The method used is the modification of that described by Gargano et al. (Exp. Gerontol. 40:386-395, 2005), to which we have added image processing that allows us to measure the climbing velocity of individual flies.

The flies to be tested were transferred into 15 ml tubes and we tapped the top of the tube. Due to their capacity for geotaxis orientation, the flies tend to climb upwards against gravity. A high speed digital imaging system/camera (Motionscope PCI, Redlake Imaging MASD, Inc.) with an attached Vivitar wide-angle lens, was used to capture video sequences at 60 frames per second of the flies as they climbed the tube. Images were analyzed with software (MotionScope 2.21.1) and for each fly within the tube we were able to obtain an individual velocity value.

The first dataset of combined interventions. We performed an initial screen of compounds for their effects on cardiac aging in Drosophila, selected for their very general effects on multiple biological functions, known low toxicity and, for some, known effects on aging in other models (Wood et al., Nature 430:686-689, 2004).

After screening 44 compounds individually at multiple doses (a total of 300 groups, each composed of 10-20 flies), we chose two doses each of four compounds for more comprehensive measurements of their combined effects on three age-related phenotypes: maximal heart rate and exercise capacity declines with age and survival. The selected compounds and doses (in the fly food) were: doxycycline, a broad spectrum antibiotic and inhibitor of mitochondrial protein synthesis (Toivonen et al., Genetics 159:241-254, 2001), with concentrations at 0.5 mg/mL and 1 mg/mL; sodium selenite, an essential trace mineral and cofactor of many metabolic enzymes (Higdon, An Evidence-Based Approach to Vitamins and Minerals [New York: Thieme], 2003), at 0.005 mg/mL and 0.0125 mg/mL; zinc sulfate, another trace mineral and cofactor of many metabolic enzymes (Higdon, An Evidence-Based Approach to Vitamins and Minerals [New York: Thieme], 2003), at 0.5 mg/mL and 1 mg/mL; and resveratrol, a phenolic antioxidant with an action on proteins linked to aging (Wood et al., Nature 430:686-689, 2004), at 0.25 mM and 0.5 mM.

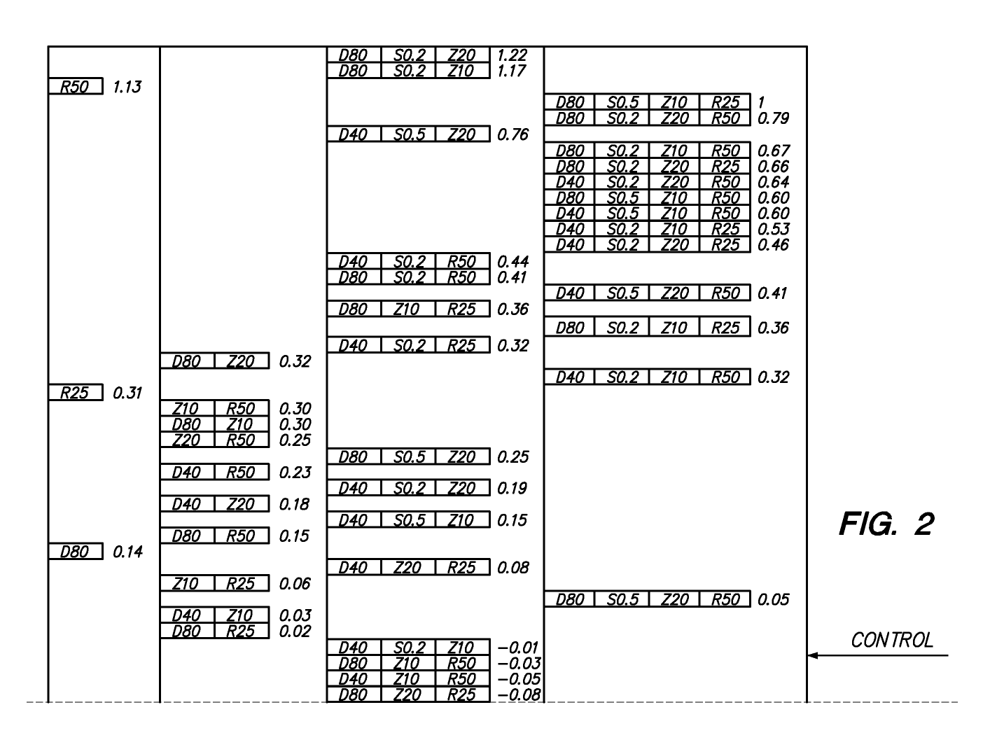

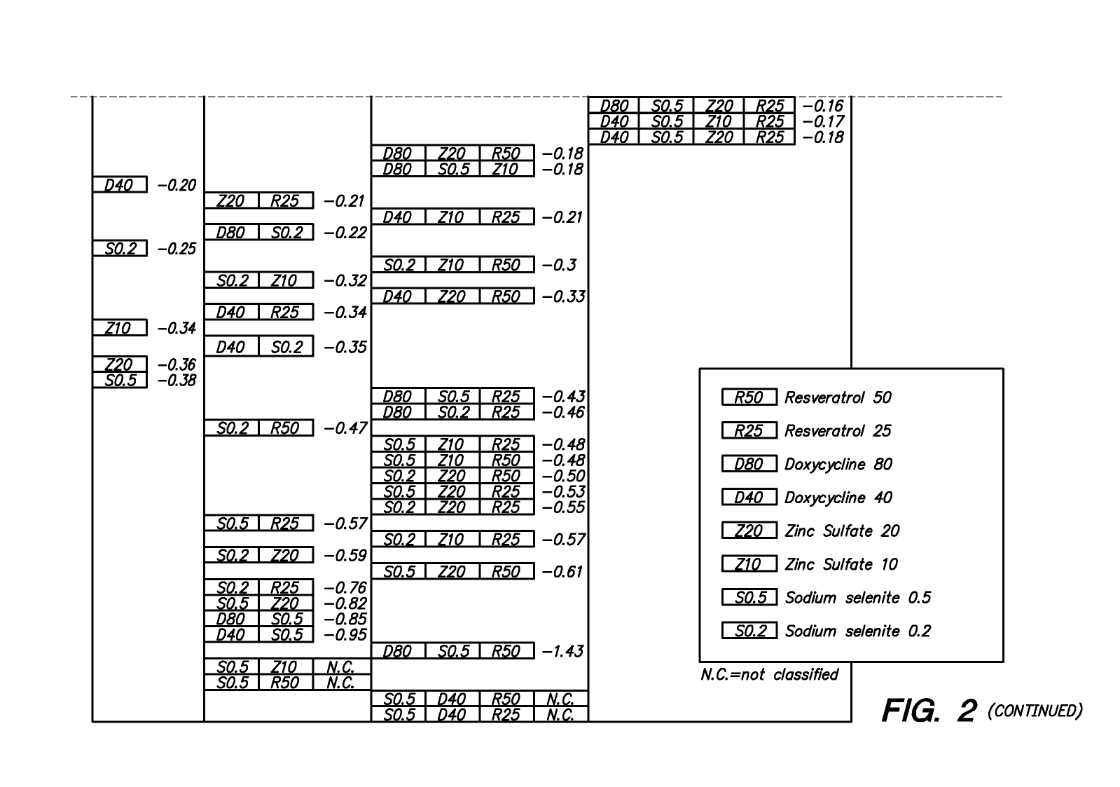

The compounds were given to flies from the age of 7 days to the age of 30 days. We have previously shown cardiac physiological changes with age in 30 day-old flies (Paternostro et al., Circulation Res. 88:1053-1058, 2001). Maximal heart rate is measured at 30 days, while climbing, a non-invasive procedure, is measured every five days from the age of 15 to the age of 30 days. We studied 10 male flies for climbing and 10 female flies for the cardiac phenotype. Survival to 30 days was also measured in these flies. While these tests had the statistical power to often detect differences in the physiological parameters, the survival measurements had mainly the purpose of detecting toxic effects, not of accurately measuring longevity (the statistical power for survival is lower because it depends on the number of deaths not on the numbers of individuals). Our main interest is in interventions that can affect quality of life in older individuals. FIG. 2 illustrates the 81 groups fully factorial dataset. Fully factorial means that all possible combinations are studied. Here we used 81 combinations of four drugs (two doses each). More in detail we have obtained measurements in one control, eight individual tests, 24 groups of two combined drugs, 32 groups of three combined drugs and 16 groups of four combined drugs.

The number on the right of each combination in FIG. 2 is a summary score (z-score) obtained from the three phenotypes we measure: maximal heart rate, exercise capacity and survival, in aged flies. We first normalize each value by dividing it by a weekly control. In order to obtain a comparable number from different measurements, for each group we subtract the mean of the entire population and we divide by the standard deviation of the entire population. The z scores from the three phenotypes are then averaged, obtaining a summary z-score that gives equal weight to each of the three measurements.

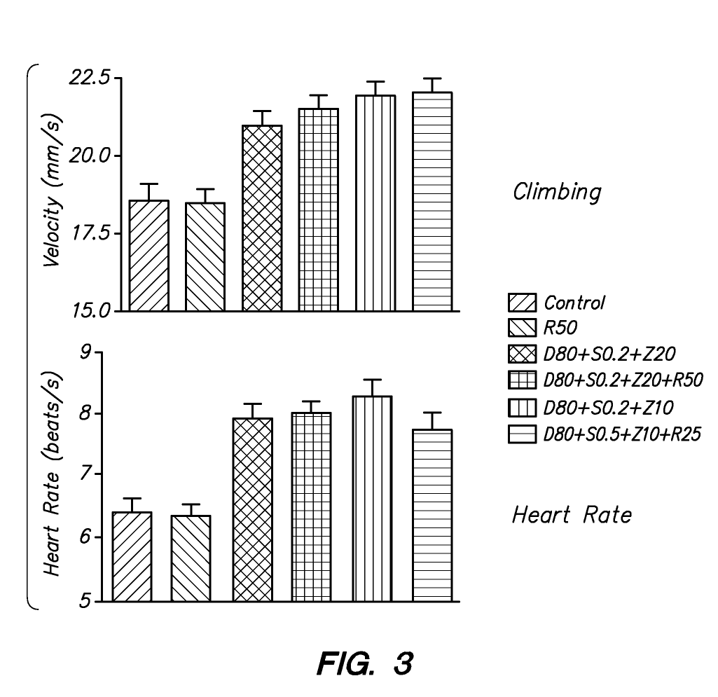

Additional tests on the five best combinations. The five best combinations from our first dataset (shown in FIG. 2) were chosen for a separate study with a larger number of tests. We studied at least 103 flies in each group for climbing velocity experiments and at least 31 flies in each group for maximal heart rate experiments. The results are shown in FIG. 3. We could confirm the functional improvement for four combinations (with high statistical significance) but not for resveratrol alone. We went back to the original data and found that in the first dataset the weekly control used for normalizing resveratrol was abnormally low. We have previously shown in Drosophila a decline with age of the maximal heart rate (Paternostro et al., Circulation Res. 88:1053-1058, 2001) and we and others (Gargano et al., Exp. Gerontol. 40:386-395, 2005) have shown a decline with age in maximal exercise capacity.

Suggested treatment protocol. The combined treatment giving the best protective effect on the decline with age of the studied physiological parameters was composed of: doxycycline, 1 mg/mL; sodium selenite, 0.005 mg/mL; and zinc sulfate, 0.5 mg/mL. These were doses in the food and we suggest a combination with the same doses in the food and water of mice. The compounds are stable and we left them in the food for 3-4 days. They are easily available commercially (for example from Sigma), and inexpensive. Mice should be treated when already adults, we suggest starting at the age of five months and continuing throughout the lifespan. Doxycycline slows development in flies.

The compounds are extremely well known pharmacologically and blood tests are probably not necessary. Physiological tests, including exercise capacity are the best way of monitoring treatment.

With regard to animal safety, the compounds have low toxicity (two are dietary supplements). A 2 mg/ml dose of doxycycline in the drinking water has been shown to be well tolerated chronically in mice and to result in plasma levels close to saturation, with no effect on body weight (Smith et al., Ann. Neurol. 54:186-196, 2003; Yamamoto et al., Cell 101:57-66, 2000). In these studies the drug was given as a 5% sucrose solution in bottles protected from light.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method of treating a symptom of aging, improving an age-related dysfunction or prolonging survival in a mammal in need of such treatment, comprising administrating to the mammal an effective amount of a composition comprising doxycycline, selenium and zinc.

2. The method of claim 1 wherein the symptom of aging is selected from the group consisting of cardiac aging and a decline in exercise capacity.

3. The method of claim 1 wherein the composition comprises about 125 μg/kg body weight per day to about 5.00 mg/kg body weight per day of doxycycline.

4. The method of claim 1 wherein the composition comprises about 0.125 μg/kg body weight per day to about 5.00 μg/kg body weight per day of selenium.

5. The method of claim 1 wherein the composition comprises about 12.5 μg/kg body weight per day to about 500 μg/kg body weight per day of zinc.

6. The method of claim 1 wherein the composition comprises about 125 μg/kg body weight per day to about 5.00 mg/kg body weight per day of doxycycline, about 0.125 μg/kg body weight per day to about 5.00 μg/kg body weight per day of selenium, and about 12.5 μg/kg body weight per day to about 500 μg/kg body weight per day of zinc.

7. The method of claim 1 wherein the mammal is a human.

8. A method of making a medicament useful in treating a symptom of aging, improving an age-related dysfunction or prolonging survival in a mammal in need of such treatment, the method comprising incorporation of doxycycline, selenium and zinc into a pharmaceutical composition comprising the doxycycline, selenium and zinc and a pharmaceutically acceptable carrier.

9. A composition for treating a symptom of aging, improving an age-related dysfunction or prolonging survival in a mammal in need of such treatment, the composition comprising doxycycline, selenium and zinc.

10. The composition of claim 9 wherein the symptom of aging is selected from the group consisting of cardiac aging and a decline in exercise capacity.

11. The composition of claim 9 wherein the composition comprises about 10 mg to about 400 mg of doxycycline.

12. The composition of claim 9 wherein the composition comprises about 10 μg to about 400 μg of selenium.

13. The composition of claim 9 wherein the composition comprises about 1 mg to about 40 mg of zinc.

14. The composition of claim 9 wherein the composition comprises about 10 mg to about 400 mg of doxycycline, about 10 μg to about 400 μg of selenium, and about 1 mg to about 40 mg of zinc.

15. The composition of claim 9 comprising a pharmaceutically acceptable carrier.
